(19) United States
(12) Patent Application Publication
(10) Pub. No.: US 2005/0281848 A1
(43) Pub. Date: Dec. 22, 2005

(54) Oleaginous ointments comprising two solubilized bioactive agents for the treatment of psoriasis

(75) Inventors: Leslie Zanutto, Paulhaguet (FR); Sandrine Orsoni, Mandelieu (FR); Nathalie Barthez, Nice (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA S.A., CHAM (CH)

(21) Appl. No.: 10/959,431

(22) Filed: Oct. 7, 2004

(30) Foreign Application Priority Data
Jun. 17, 2004 (FR) 04/06609

(51) Int. Cl. A61K 31/59; A61K 31/573

(52) U.S. Cl. 424/400; 514/167; 514/171

(57) ABSTRACT

Topically applicable, anhydrous, hydrophobic and physically/chemically stable dermatological/pharmaceutical oleaginous ointments having effective release/penetration capacity and also having therapeutically effective amounts of calcitriol and clobetasol 17-propionate solubilized therein, are well suited for the treatment of psoriasis.

### CROSS-REFERENCE TO PRIORITY APPLICATION

This application claims priority under 35 U.S.C. § 119 of FR 04/06609, filed Jun. 17, 2004, hereby expressly incorporated by reference and assigned to the assignee hereof.

### BACKGROUND OF THE INVENTION

1. Technical Field of the Invention

The present invention relates to the field of the formulation of bioactive principles for the purpose of topical pharmaceutical application.

The present invention relates more particularly to stable, anhydrous dermatological/pharmaceutical compositions comprising oleaginous ointments and as active principles calcitriol and clobetasol 17-propionate, to the process for preparing same and to the treatment of psoriasis and other skin disorders via topical administration thereof.

2. Description of Background and/or Related and/or Prior Art

Calcitriol is an analogue of vitamin D and is administered in particular to regulate the level of calcium in the body. Vitamin D and its derivatives are generally used in dermatology in the treatment of psoriasis since they limit the excessive production of skin cells on the surfaces affected and possess proven advantages for the treatment of this ailment, which is characterized in particular by the presence of thick, squamous and dry lesions.

Clobetasol 17-propionate is a corticosteroid. The mechanism of action of corticosteroids is attributed to their inhibition of inflammatory processes (Lange K et al., Skin Pharmacol Appl Skin Physiol, 13(2): 93-103 (2000)).

U.S. Pat. No. 4,610,978 describes compositions for topical application in the treatment of dermatological diseases such as, for example, psoriasis and comprising calcitriol. These compositions may additionally contain a corticosteroid such as, for example, hydrocortisone or dexamethasone. WO 00/64450 and WO 02/34235 describe pharmaceutical compositions for dermal use that contain vitamin D or a vitamin D analogue and a corticosteroid. The examples given relate more particularly to compositions comprising calcipotriol (a vitamin D analogue) and betamethasone (a corticosteroid). Comparing measurements of efficacy on patients affected by psoriasis for a composition containing calcipotriol alone, betamethasone alone or the combination of the two actives shows that the effect obtained by means of the combination corresponds to an additive effect. Thus, from this prior art, one skilled in the art could not have considered that the combination of a vitamin D analogue with a corticosteroid might exhibit a synergistic effect. Furthermore, the aforesaid does not specifically describe the combination of clobetasol propionate with calcitriol.

FR-2,848,454, assigned to the assignee hereof, describes that the combination of calcitriol with clobetasol propionate made it possible to obtain a synergistic effect in the treatment of certain dermatological ailments such as psoriasis, atopic dermatitis, contact dermatitis and seborrheic dermatitis.

However, the combination in a single pharmaceutical composition of calcitriol with clobetasol propionate is not without certain problems. The reason for this is that the calcitriol is unstable in aqueous media and more particularly so at acidic pH values, whereas clobetasol 17-propionate is unstable in a basic environment.

Consequently, it is appropriate to formulate these active principles in anhydrous compositions. The anhydrous compositions presently available which allow the formulation of water-sensitive active principles while providing them with effective chemical stability are generally ointment compositions. These ointment compositions are composed primarily of petroleum jelly, mineral oil and/or vegetable oil. The treatments currently on the market, however, either include a high percentage of petroleum jelly, in order to promote the occlusiveness and the penetration of the active, or contain a high percentage of glycol penetration promoter, in order to promote the penetration of the active, but are sticky and may give rise to problems of intolerance (see article “The critical role of the vehicle to therapeutic efficacy and patient compliance”, Piacquadio et al, J. Am. Acad. Dermatol., August 1998).

The ointment compositions currently on the market do not always lend themselves to the formulation of the active principle in a solubilized form.

### SUMMARY OF THE INVENTION

The present invention features anhydrous pharmaceutical compositions suited for topical application and which ameliorate or avoid the aforementioned drawbacks and disadvantages of the prior art.

The present invention, thus, also features anhydrous pharmaceutical compositions suited for topical application and whose active principles are in solubilized form and exhibit prolonged stability.

This invention also features anhydrous pharmaceutical compositions suited for topical application and which exhibit very good tolerance.

The present invention accordingly features anhydrous pharmaceutical compositions suited for the treatment of psoriasis, which comprise an oleaginous ointment and as active principles calcitriol and clobetasol 17-propionate, said actives each being in a solubilized form in the said composition.

### DETAILED DESCRIPTION OF BEST MODE AND SPECIFIC/PREFERRED EMBODIMENTS OF THE INVENTION

Advantageously, the amount of calcitriol in solubilized state in the compositions of the invention is from 0.00001 to 5% by weight relative to the total weight of the composition, preferably from 0.0001 to 3% by weight and more particularly from 0.0003 to 1% by weight.

The amount of clobetasol 17-propionate in solubilized form is from 0.0001 to 3% by weight relative to the total weight of the composition, preferably from 0.00005 to 1% by weight and more particularly from 0.001 to 0.1% by weight.

The compositions of the invention are more particularly suited for topical application.

The active principles comprising the compositions of the invention, namely calcitriol and clobetasol 17-propionate, are in the solubilized state so as to endow the subject compositions with good skin penetration/release properties for each of the said active principles, in conjunction with more advantageous kinetics. The term “effective release/penetration capacity” refers to effective distribution of the composition of the invention and therefore of the active principles it contains across the stratum corneum of the skin and across the subcutaneous layers such as the epidermis and the dermis.

The expression “anhydrous composition” refers for the purposes of the present invention to a composition which is substantially devoid of water, in other words having a water content of less than or equal to 5% by weight relative to the total weight of the composition, in particular less than or equal to 3%, and more particularly zero.

For the purposes of the present invention and in accordance with the US Pharmacopeia (USP) the term “ointment” refers to a semisolid preparation which is intended for external application to the skin or mucosae. Ointments or unguents are used topically for numerous applications, for example as barrier creams, antiseptic creams, emollient creams, etc. Ointments are used for their emollient effect; they are easy to apply and readily penetrate the skin.

There are commonly five types of ointments, differentiated on the basis of their physical composition. The most common type of ointment, which is that to which the present invention relates, is the ointment with an oleaginous base, referred to as “oleaginous ointment”; this ointment is anhydrous and hydrophobic and comprises predominantly petroleum jelly and/or liquid paraffins. The other constituents of the ointment may be:

oils of mineral, animal, vegetable or synthetic origin;

a thickener such as waxes, of mineral, vegetable and animal origin;

butters: cocoa butter, karite butter, copra butter;

lanolin and its derivatives.

Petroleum jelly (petrolatum) is a mixture of long-chain aliphatic hydrocarbons and is an excellent moisturizer. This is because its occlusion properties allow the imperceptible transcutaneous loss of water to be blocked and the water to be trapped under the surface of the skin, by virtue of the formation of an inert occlusion membrane (“Effects of petrolatum on stratum corneum structure and function” Ghadially & all; Journal of the American Academy of Dermatology 1992; 26: 387-96). Petroleum jelly accelerates the recovery of the normal skin barrier properties in the case of skin affected by lesions, such as in atopic dermatitis or psoriasis, for example. Petroleum jelly alone, owing to its emollience, is a good moisturizer and may attenuate psoriatic plaques. Petroleum jelly, moreover, is inert and therefore has no incompatibility at all, irrespective of the active principle or principles.

Ointments based on petroleum jelly are recognized as being among the best systems for release of active agents in terms of efficacy. This is because the occluded skin becomes more permeable and the active principles are better able to penetrate. It is also known that the efficacy of topical steroids may be enhanced by application of an occlusive film created by the ointment (“Hospital practice: current treatment options in psoriasis” Khachemoune et al, 2000, pp. 1-13).

As an example of mineral oil, mention may be made of the liquid paraffins of various viscosities such as Primol 352, Marcol 82 and Marcol 152 which are sold by Esso.

As a vegetable oil, mention may be made of sweet almond oil, palm oil, soya oil, sesame oil and sunflower oil.

As an animal oil, mention may be made of lanolin, squalene, fish oil and mink oil.

As a synthetic oil, mention may be made of an ester such as cetearyl isononanoate, such as the product sold under the name Cetiol SN by Cognis France, diisopropyl adipate such as the product sold under the name Ceraphyl 230 by ISF, isopropyl palmitate such as the product sold under the name Crodamol IPP by Croda, and caprylic/capric triglyceride such as Miglyol 812, sold by Huls/Lambert Rivière.

The term “wax” refers generally to a lipophilic compound which is solid at ambient temperature (25° C.), having a reversible solid/liquid state change, and has a melting point which is greater than or equal to 30° C. and may range up to 200° C. and in particular up to 120° C.

Waxes useful in the compositions according to the invention are selected from the group consisting of waxes of animal, vegetable, mineral or synthetic origin and mixtures thereof.

According to one particular embodiment, hydrocarbon wax may be selected from the glyceryl esters of saturated and unsaturated acids, especially polyunsaturated acids, having in particular from 10 to 24 carbon atoms, unsaturated fatty acids and in particular from polyunsaturated fatty acids.

As hydrocarbon waxes of the type which are esters of glycerides and of polyunsaturated fatty acids and can be used in the compositions according to the invention, mention may be made in particular of the atomized glyceryl dipalmitostearate (C₁₆-C₁₈) sold under the name Précirol ATO 5® by Gattefosse, the atomized glyceryl behenate (C₂₂) sold for example under the name Compritol® by Gattefosse, and mixtures thereof.

It is also possible to use hydrocarbon waxes such as beeswax, lanolin wax and China insect waxes; rice wax, carnauba wax, candelilla wax, ouricury wax, Alfa wax, cork fiber wax, sugarcane wax, Japan wax and sumac wax; montan wax, microcrystalline waxes, paraffins and ozokerite; polyethylene waxes, waxes obtained by Fischer-Tropsch synthesis and waxy copolymers, and also esters thereof.

Mention may also be made of waxes obtained by catalytic hydrogenation of animal or vegetable oils having C₈-C₃₂, linear or branched fatty chains.

Among these waxes, mention may be made in particular of hydrogenated jojoba oil, isomerized jojoba oil such as the trans-isomerized, partially hydrogenated jojoba oil manufactured or sold by Desert Whale under the commercial reference ISO-JOJOBA-50®, hydrogenated sunflower oil, hydrogenated castor oil, hydrogenated copra oil and hydrogenated lanolin oil, the di(1,1,1-trimethylolpropane)tetrastearate sold under the name Hest 2T-4S by Heterene, and the di(1,1,1-trimethylolpropane)tetrabehenate sold under the name Hest 2T-4B by Heterene.

Mention may also be made of silicone waxes and fluoro waxes.

It is also possible to use the wax obtained by hydrogenating esterified olive oil with stearyl alcohol that is sold under the name Phytowax Olive 18 L 57 or else waxes obtained by hydrogenating esterified castor oil with cetyl alcohol, these waxes being sold under the name Phytowax ricin 16L64 and 22L73 by Sophim. Such waxes are described in FR-A-2,792,190.

The thickener may be selected from butters and lanolin.

According to one preferred embodiment of the invention, the thickener is beeswax, hydrogenated castor oil, carnauba oil, alkylmethylsiloxane wax (ST wax 30) or candelilla wax.

The additional thickener content depends, as will be appreciated, on the viscosity of the desired composition. It can of course be determined by one skilled in the art employing simple, routine operations.

Generally speaking, the amount of additional thickener, and in particular of pasty or solid hydrocarbon compound, is from 2 to 20% by weight relative to the total weight of the composition, in particular from 5 to 10%.

Karite butter (shea butter) is a vegetable fatty material which is used traditionally on the African continent, as a basic product of African pharmacology. By way of example, African newborns are rubbed with karite butter from birth in order to protect them from extreme climatic conditions. Karite butter has been used by cosmetics chemists for more than 20 years. It is a natural fat obtained from the karite tree (Butyrospermum parkii). Chemical analysis of karite butter reveals it to be composed of fatty acids and their glycerides. The fatty acids present in karite butter are saturated and unsaturated. Karite butter is known in particular for:

its calming and softening virtues on dry and sensitive skin,

its decongestant effects,

its efficacy in reducing the signs of aging

its scar-forming and regenerative action,

its moisturizing action, and

its antierythematous UV protection (in animals).

Furthermore, karite butter has excellent tolerance.

According to one advantageous embodiment of the invention, the components of the oleaginous ointment are more particularly selected from the group consisting of petroleum jelly and/or liquid paraffin, karite butter and additionally an emollient.

The action of an emollient product is to render the skin supple and smooth and to promote its well-being. Emollient action takes the form either of moisturizing of the stratum corneum or by compensation for the insufficiency of sebaceous secretion.

The stratum corneum may be moisturized in a number of ways: using substances which slow down dehydration by virtue of an occlusive effect (fats: waxes, oils, fatty alcohols, silicone oils) or the use of humectants (polyols, glycerol, urea).

Insufficiency of sebaceous secretion is for its part compensated by the use of lipid products such as oils.

According to another advantageous embodiment of the invention, the active principles are solubilized in a single solvent or in two or more solvents.

The solvent of the present invention is selected from pharmaceutically acceptable compounds, in other words compounds whose use is particularly compatible with application to the skin, mucosae and/or keratin fibers. It is generally fluid, and in particular liquid, at ambient temperature and under atmospheric pressure.

As solvents according to the invention, mention may be made in particular of the following:

propylene glycol,

oils such as caprylic and capric triglycerides (Miglyol 812), cetearyl isononanoate (Cetiol SN) and vegetable oils (sweet almond oil, sesame oil, sunflower oil, etc.) and mixtures thereof, and

mixtures thereof.

The solvent is generally present in the compositions of the invention in an amount which on the one hand is sufficient to provide the required solubility of the active principles to be formulated and on the other hand which is compatible with the need to preserve prolonged chemical stability of these same active principles. In other words, the solvent must be chemically inert with respect to the active principles.

Advantageously, the amount of solvent for each of the actives in a composition of the invention is from 10 to 30% by weight relative to the total weight of the composition, preferably from 5 to 20% by weight.

The solvent likewise confers a beneficial effect on the skin penetration rate of the active principles.

The compositions according to the invention may further comprise various other ingredients. The choice of these supplementary ingredients, and the choice of their respective amounts, is made so as not to prejudice the expected properties of the composition. In other words, these compounds must adversely affect neither the chemical stability of the active principles nor their solubility.

The compositions of the invention may further comprise a lipophilic anti-irritant. By way of example, mention may be made of DL-alpha-tocopherol acetate, oil of Melaleuca alternifolia, green tea extract, calendula extract and karite butter.

According to another advantageous embodiment, the compositions of the invention may further comprise an antioxidant selected from the group consisting of butylated hydroxytoluene (BHT), butylated hydroxyanisole (BHA), DL-alpha-tocopherol and propyl gallate.

According to one preferred embodiment, the composition of the invention is an anhydrous pharmaceutical ointment composition comprising petroleum jelly and as actives calcitriol and clobetasol 17-propionate in solubilized form.

A composition of this kind is physically and chemically stable, is synergistically effective on psoriasis and allows optimized release of the two actives while at the same time allowing very good tolerance.

In addition to the actives and the petroleum jelly, one preferred composition of the invention further comprises karite butter and an emollient such as caprylic/capric triglycerides or cetearyl isononanoate or a vegetable oil and propylene glycol.

The combination of the petroleum jelly, karite butter and an emollient ensures the very good tolerance of the compositions of the invention while allowing restoration of the skin barrier that has been altered by the pathology of the psoriasis.

The compositions of the invention are found particularly effective in conserving satisfactory chemical stability of active principles which are sensitive to oxidation, to water and to aqueous media in general. The term “satisfactory chemical stability” refers to a composition which over a period of at least 3 months, respectively at ambient temperature and at 40° C.:

shows no substantial modification in its macroscopic appearance,

has an active principles content of at least 80%, in particular at least 90% and more particularly at least 95% of the initial weight content.

The present invention further provides for the formulation of an oleaginous ointment for preparing an anhydrous pharmaceutical composition useful in a regime or regimen for the treatment of psoriasis, the said composition comprising as active principles calcitriol and clobetasol 17-propionate, each of the said active principles being in a solubilized form in the said composition.

The invention provides for the administration as defined above wherein the composition is as defined above.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLE 1

### Study of Local Tolerance of a Composition of the Invention

A tolerance study was carried out on the formulation vehicle (without actives) of the invention in comparison with a vehicle known for its great tolerance (Daivonex®).

Treatment: one application daily from day 1 to day 6 of 20 μl of composition is made to the right ear of mice.

Evaluation method: clinical observation and measurement of the thickness of the mouse ear from day 2 to day 12. Weighing of the animals on day 1 and on day 12.

Conclusion:

After 12 days the vehicle of the invention is found to be non-irritant and to be comparable with the Daivonex® vehicle and with that of the untreated group.

### EXAMPLE 2

### Solubility and Stability of the Compositions of the Invention:

The stability of calcitriol was evaluated in the following two oil solvents: Miglyol 812 and Cetiol SN.

a) Stability of Calcitriol in Miglyol 812 (Caprylic and 20 Capric Triglycerides):

A solution is prepared of calcitriol 30 ppm in qs 100% of Miglyol 812 in the presence of 0.4% of BHT (antioxidant), kept under nitrogen.

The HPLC assay technique results against a reference are indicated in Table 1 below.

The initial time (TO) is taken as 100%.

TABLE 1

| Stability conditions | T2 weeks | T4 weeks |
|---|---|---|
| +4° C. | 98.2% | 105.2% |
| Ambient temperature | 95.8% | 98.0% |
| +40° C. | 93.1% | 95.0% |

Calcitriol is stable for 1 month in solution in caprylic/capric triglyceride

b) Stability of Calcitriol in Cetiol SN (Cetearyl Isononanoate):

A solution is prepared of calcitriol 30 ppm in qs 100% of Cetiol SN in the presence of 0.4% of BHT and kept under nitrogen.

The HPLC assay technique results against a reference are indicated in Table 2 below.

The initial time (TO) is taken as 100%.

TABLE 2

| Stability conditions | T2 weeks | T4 weeks |
|---|---|---|
| +4° C. | 103.7% | 97.9% |
| AT | 99.6% | 98.7% |
| +40° C. | 99.4% | 98.1% |

Calcitriol is stable for 1 month in solution in cetearyl isononanoate.

### EXAMPLE 3

### Preparation of a Composition of the Invention

The invention relates to an anhydrous formulation which allows all of the constituents to be incorporated at a high temperature at which the petroleum jelly is liquid, and therefore allows effective mixing of the constituents. This also makes it possible to obtain effective stability at 30° C. without any exudate.

The process is performed in a water bath, which allows a homogeneous temperature to be maintained throughout preparation; in addition it is also important to cover the formulating beaker in order to prevent any crusting. The process is performed with the aid of a butterfly blade, which allows effective circulation within pasty products, thereby ensuring effective homogenization.

a) First Step: Preparation of the Fatty Phase Comprising the Oleaginous Ointment:

The petroleum jelly (oleaginous ointment), the thickener and the lipophilic anti-irritant are weighed out into a beaker.

The beaker is heated at 75° C. in the water bath with gentle Rayneri (butterfly blade) stirring.

b) Second Step: Preparation of the Phase Comprising the Active Principles:

A stock solution of calcitriol is prepared in the appropriate solvent, an antioxidant is added and the mixture is stirred until the active is solubilized.

Clobetasol 17-propionate and its solvent are weighed out and the mixture is stirred until the active is solubilized.

These two active phases are incorporated into the fatty phase prepared as above at 75° C. with Rayneri stirring.

The mixture is homogenized and the composition is allowed to cool to 30° C. in the water bath, with Rayneri stirring.

Packaging is carried out at 30° C., a temperature at which the composition has not yet completely solidified.

### EXAMPLE 4

### Examples of Compositions in Accordance With the Invention:

TABLE 3
Composition 1

| | Amounts in % weight |
|---|---|
| Raw materials | for weight |
| WHITE PETROLATUM | qs 100 |
| CARNAUBA WAX | 9 |
| CAPRYLIC/CAPRIC TRIGLYCERIDE | 10 |
| dl-ALPHA-TOCOPHEROL ACETATE | 1 |
| CALCITRIOL | 0.0009 |
| CLOBETASOL PROPIONATE | 0.01 |
| PROPYLENE GLYCOL | 10 |

TABLE 4
Composition 2

| | Amounts in % weight for |
|---|---|
| Raw materials | weight |
| WHITE PETROLATUM | qs 100 |
| BEESWAX | 5 |
| CAPRYLIC/CAPRIC TRIGLYCERIDE | 15 |
| SHEA BUTTER | 5 |
| BUTYLATED HYDROXYTOLUENE | 0.04 |
| CALCITRIOL | 0.0003 |
| CLOBETASOL PROPIONATE | 0.025 |
| PROPYLENE GLYCOL | 10 |

The physical stability of compositions 1 and 2 is measured by macroscopic and microscopic observation of the composition at ambient temperature, at 4° C. and at 30° C. after 15 days, 1 month, 2 months and 3 months.

At ambient temperature macroscopic observation allows the physical integrity of the products to be guaranteed and microscopic observation makes it possible to verify that there is no recrystallization of the solubilized actives.

At 4° C. microscopic observation verifies the non-recrystallization of the solubilized actives.

At 30° C. macroscopic observation verifies the integrity of each of the end compositions.

The characterization of each of the end compositions is completed by a measurement of the flow point. A Haake VT550 rheometer is used with an SVDIN measuring spindle. The rheograms are performed at 25° C. and at a shear rate of 4 s⁻¹ (γ), measuring the shearing stress. By flow point (τ0, expressed in pascals) is meant the force required (minimum shearing stress) to overcome the van der Waals cohesion forces and to bring about flow. The flow point is taken to be the value found at a shear rate of 4 s⁻¹.

These measurements are performed at T0, after 1 month, 2 months and 3 months.

COMPOSITION 2:
SPECIFICATIONS AT T0

| Tau 0 | | 219 | Macroscopic | Shiny, pale |
|---|---|---|---|---|
| | | | appearance | yellow ointment |
| Centrifugation | 3000 | RAS | Microscopic | Numerous |
| | rpm | | appearance | yellow, violet |
| | | | | and blue |
| | | | | refringencies |
| | | | | characteristic of |
| | 10,000 | Exudate | | the petroleum |
| | rpm | | | jelly network |

PHYSICAL STABILITY:

| | | | T15 d |
|---|---|---|---|
| AT | Macroscopy | | COMPLIES |
| | Microscopy | | COMPLIES |
| | Tau 0 | | 230 |
| | Centrifugation | 3000 | COMPLIES |
| | | 10000 | COMPLIES |

| | | |
|---|---|---|
| 4° C. | Macroscopy | COMPLIES |
| | Microscopy | COMPLIES |
| 30° C. | Macroscopy | COMPLIES |
| | Microscopy | COMPLIES |
| | Tau 0 | NR |

CHEMICAL STABILITY:

| | | | |
|---|---|---|---|
| Chemical | T0 | AT | 40° C. |
| STABILITY | | CALCITRIOL: 97.6% | CALCITRIOL: / |
| | | CLOBETASOL | CLOBETASOL |
| | | PROPIONATE: 97.3% | PROPIONATE: / |
| | T15 d | AT | 40° C. |
| | | CALCITRIOL: 95.1 | CALCITRIOL: 86.8 |
| | | CLOBETASOL | CLOBETASOL |
| | | PROPIONATE: 95.2 | PROPIONATE: 92.3 |

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

1. A topically applicable, anhydrous, hydrophobic and physically/chemically stable dermatological/pharmaceutical oleaginous ointment having effective release/penetration capacity and also having therapeutically effective amounts of calcitriol and clobetasol 17-propionate solubilized therein.

2. The anhydrous, stable oleaginous ointment as defined by claim 1, having a water content of less than or equal to 5% by weight.

3. The anhydrous, stable oleaginous ointment as defined by claim 1, having a water content of less than or equal to 3% by weight.

4. The anhydrous, stable oleaginous ointment as defined by claim 1, having a water content of zero.

5. The anhydrous, stable oleaginous ointment as defined by claim 1, comprising petroleum jelly and/or liquid paraffin.

6. The anhydrous, stable oleaginous ointment as defined by claim 1, comprising at least one emollient.

7. The anhydrous, stable oleaginous ointment as defined by claim 1, further comprising karite butter.

8. The anhydrous, stable oleaginous ointment as defined by claim 1, said calcitriol and said clobetasol 17-propionate being solubilized in a single solvent.

9. The anhydrous, stable oleaginous ointment as defined by claim 1, said calcitriol and said clobetasol 17-propionate being solubilized in two or more different solvents.

10. The anhydrous, stable oleaginous ointment as defined by claim 8, comprising a solvent selected from the group consisting of propylene glycol, an oil, and mixtures thereof.

11. The anhydrous, stable oleaginous ointment as defined by claim 10, comprising an oil solvent selected from the group consisting of caprylic and capric triglycerides, cetearyl isononanoate, vegetable oils, sweet almond oil, sesame oil, sunflower oil, and mixtures thereof.

12. The anhydrous, stable oleaginous ointment as defined by claim 9, comprising at least two solvents selected from the group consisting of propylene glycol, an oil, and mixtures thereof.

13. The anhydrous, stable oleaginous ointment as defined by claim 12, comprising at least one oil solvent selected from the group consisting of caprylic and capric triglycerides, cetearyl isononanoate, vegetable oils, sweet almond oil, sesame oil, sunflower oil, and mixtures thereof.

14. The anhydrous, stable oleaginous ointment as defined by claim 1, comprising from 0.00001 to 5% by weight of solubilized calcitriol.

15. The anhydrous, stable oleaginous ointment as defined by claim 1, comprising from 0.0001 to 3% by weight of solubilized calcitriol.

16. The anhydrous, stable oleaginous ointment as defined by claim 1, comprising from 0.003 to 1% by weight of solubilized calcitriol.

17. The anhydrous, stable oleaginous ointment as defined by claim 14, comprising from 0.0001 to 3% by weight of solubilized clobetasol 17-propionate.

18. The anhydrous, stable oleaginous ointment as defined by claim 14, comprising from 0.00005 to 1 % by weight of solubilized clobetasol 17-propionate.

19. The anhydrous, stable oleaginous ointment as defined by claim 14, comprising from 0.001 to 0.1% by weight of solubilized clobetasol 17-propionate.

20. The anhydrous, stable oleaginous ointment as defined by claim 8, comprising from 10 to 30% by weight of solvent.

21. The anhydrous, stable oleaginous ointment as defined by claim 8, comprising from 5 to 20% by weight of solvent.

22. The anhydrous, stable oleaginous ointment as defined by claim 9, comprising from 10 to 30% by weight of solvent.

23. The anhydrous, stable oleaginous ointment as defined by claim 8, comprising from 5 to 20% by weight of solvent.

24. The anhydrous, stable oleaginous ointment as defined by claim 1, further comprising at least one thickener.

25. The anhydrous, stable oleaginous ointment as defined by claim 24, said at least one thickener comprising a solid or pasty hydrocarbon, a hydrocarbon wax, a butter, lanoline, or mixture thereof.

26. The anhydrous, stable oleaginous ointment as defined by claim 1, further comprising at least one lipophilic anti-irritant.

27. The anhydrous, stable oleaginous ointment as defined by claim 1, further comprising at least one antioxidant.

28. The anhydrous, stable oleaginous ointment as defined by claim 1, further comprising at least one topically applicable pharmaceutical excipient.

29. A regime or regimen for the treatment of psoriasis, comprising topically applying onto the affected area of the skin of an individual afflicted with psoriasis, of a topically applicable, anhydrous, hydrophobic and physically/chemically stable dermatological/pharmaceutical oleaginous ointment having effective release/penetration capacity and also having therapeutically effective amounts of calcitriol and clobetasol 17-propionate solubilized therein.
